(19) United States
(12) Patent Application Publication
(10) Pub. No.: US 2016/0100986 A1
(43) Pub. Date: Apr. 14, 2016

(54) WOUND CARE DEVICE

(71) Applicant: Lawrence M. RINKE, Palm Beach Gardens, FL (US)

(72) Inventor: Lawrence M. RINKE, Palm Beach Gardens, FL (US)

(21) Appl. No.: 14/509,544

(22) Filed: Oct. 8, 2014

(51) Int. Cl. A61F 13/00

(52) U.S. Cl. CPC A61F 13/00021; A61F 13/00063; A61F 2013/00093; A61F 2013/00144; A61F 2013/00148; A61F 2013/00272

(57) ABSTRACT

A wearable article formed of a gelatinous elastomer composition having 100 parts by weight of triblock copolymer of the general configuration poly(styrene-ethylene-butylene-styrene) wherein the styrene end block to ethylene and butylene center block ratio is within the range of from between 31:69 to 40:60, from about 300 to about 1,600 parts by weight of a plasticizing oil and having a gel rigidity of about 20 gram to about 700 gram Bloom.

### CROSS REFERENCE TO RELATED APPLICATION

This application is a continuation of copending application Ser. No. 13/564,785, filed Aug. 2, 2012, which was a continuation of application Ser. No. 13/121,980, filed on Jun. 10, 2011,which was the National Stage of International Application No. PCT/US2009/055356, filed Aug. 28, 2009, which claims the benefit of U.S. Provisional Application No. 61/101,700, filed Oct. 1, 2008.

### BACKGROUND OF THE INVENTION

The present invention relates to a wound care device. More specifically, the present invention relates to a device for maintaining dressings, bandages, medical devices, or hot/cold compresses in place.

Typically, adhesive tape is the means most commonly used to secure wound dressings to a patient. The primary problem associated with this is the fact that significant numbers of patients, particularly the elderly and diabetics along with burn victims, have sensitive skin that is damaged by the repeated application and removal of tape during the healing process. This results in a longer, more expensive recovery for these patients, because the tape-related wounds also require treatment. Accordingly, wound care specialists advocate the use of a tapeless dressing system in the treatment of such patients.

The medical/surgical binders and wraps currently available to the healthcare industry exhibit a variety of shortcomings. Some may, for example, be constructed primarily of materials that trap and hold body heat or may affix themselves to the wound. Others may be made of relatively inelastic, more rigid materials that do not readily conform to the natural contours of the human anatomy. These products are not well tolerated by most patients for continuous dressing support.

Additionally, most of the wound stabilizers or wound retentive devices require a fixing device that maintains the device about the wound. Many of the retaining devices are formed of some sort of woven or netting material. For example, some materials currently used include a non-woven material that can stretch and does not include latex. Such a material can be formed into a panel that can include a stretchable web and has discrete strips of elastomeric material, which often move and fail to remain in the proper position. The panel can be placed directly on the wound prior to applying a cover and it can be wound about dressings covering the wound. The material includes a re-closable fastener system that secures the edges together and thus is not a single unit that can be stretched about the wound or area for maintaining the dressing in place.

### SUMMARY OF THE INVENTION

The present invention provides an article formed of a material that is resilient, pliable, and non-abrasive. The material can be formed into a wound care device or an article of clothing or for health and fitness purposes. The material can be formed by injection molding or extrusion. The present invention also provides a retentive device that maintains another item in place without the use of adhesives or fasteners.

These and other objects, advantages, and features of the invention will be more fully understood and appreciated by reference to the description of the current embodiment and the drawings.

### BRIEF DESCRIPTION OF THE DRAWINGS

FIG. 1 shows several embodiments for the device of the present invention.

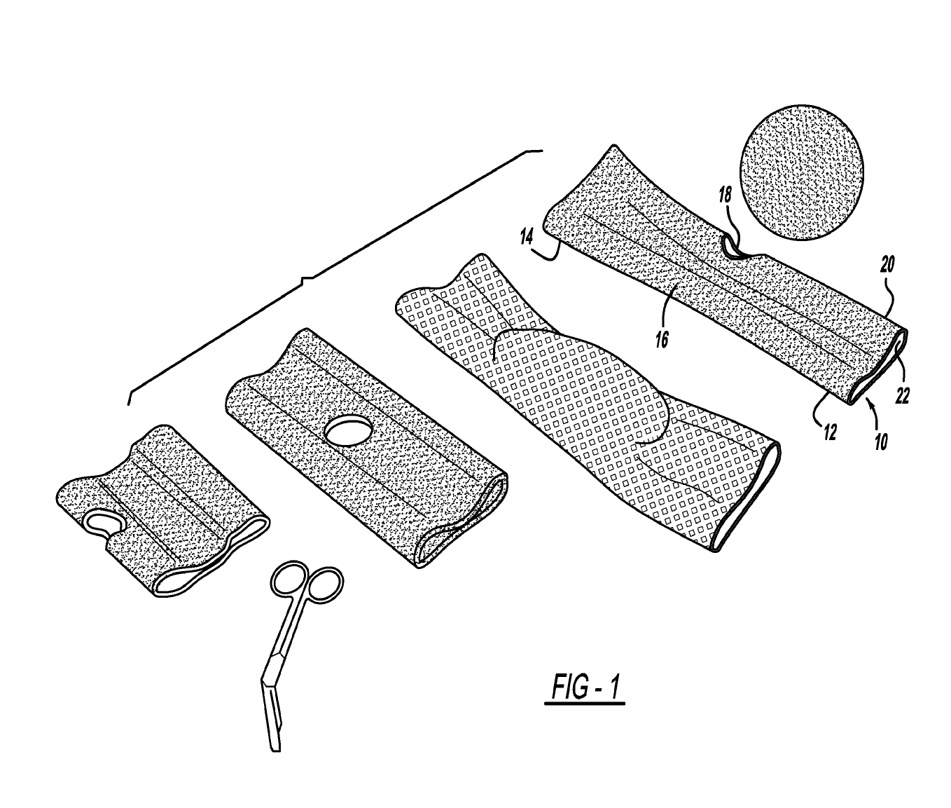

FIGS. 2A and 2B show a top view, and cross section view, respectively, of one embodiment of the device of the present invention.

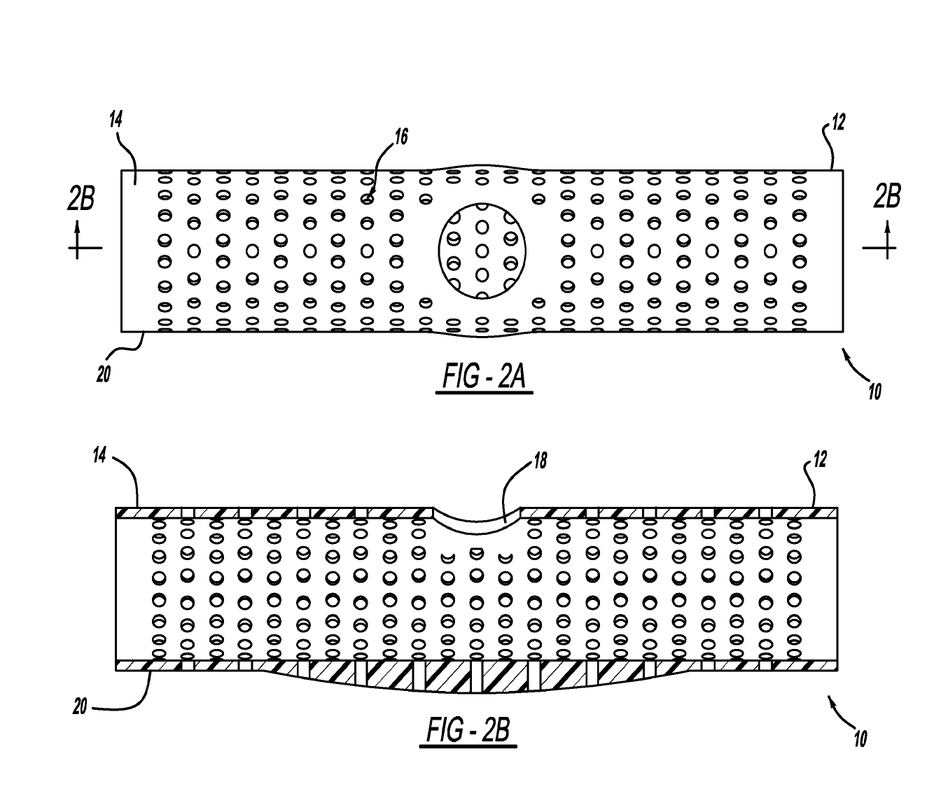

FIG. 3 is a bottom view of one embodiment of the device of the present invention.

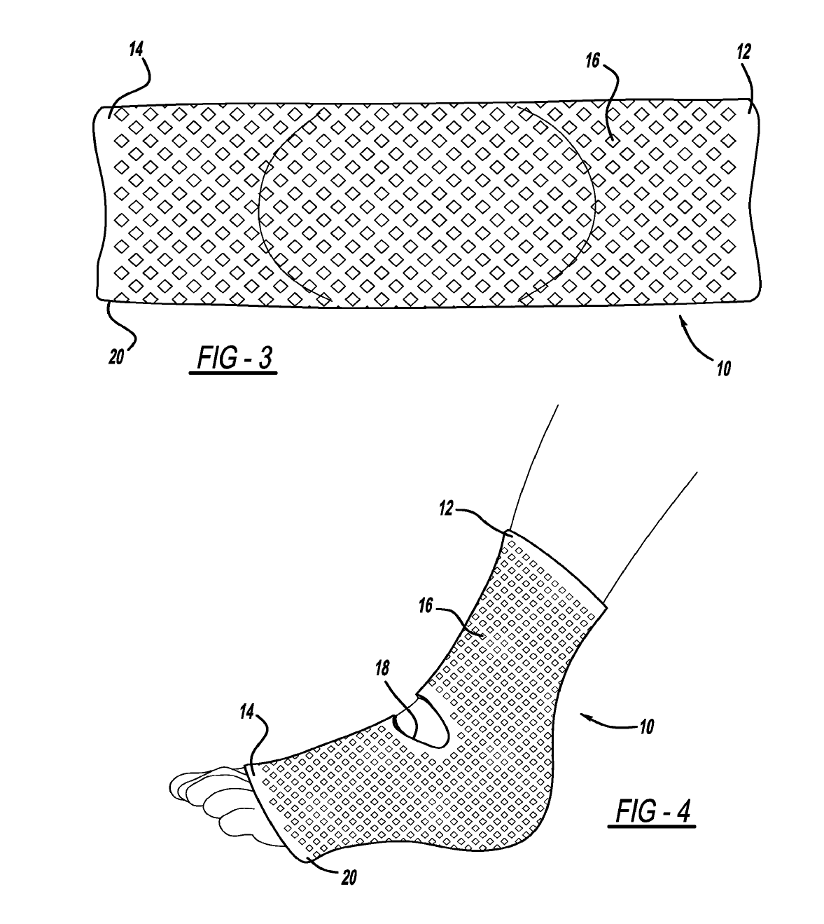

FIG. 4 is a side view of one embodiment of the device of the present invention with a foot contained within the device.

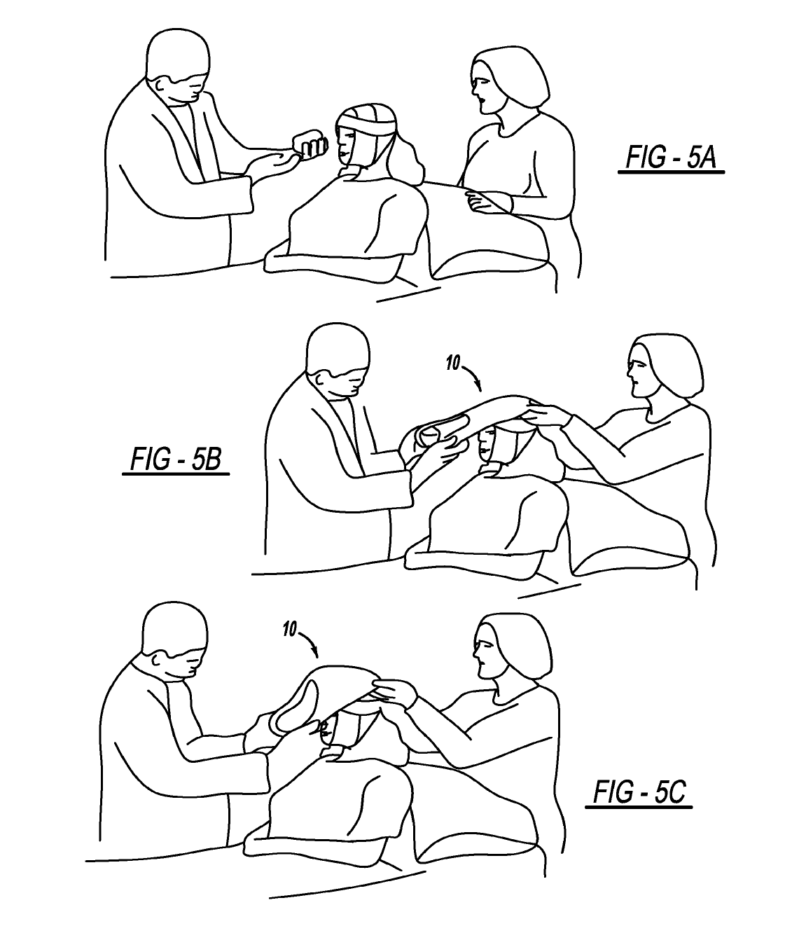

FIGS. 5A-E are consecutive views showing the device of the present invention being placed about a patient's head.

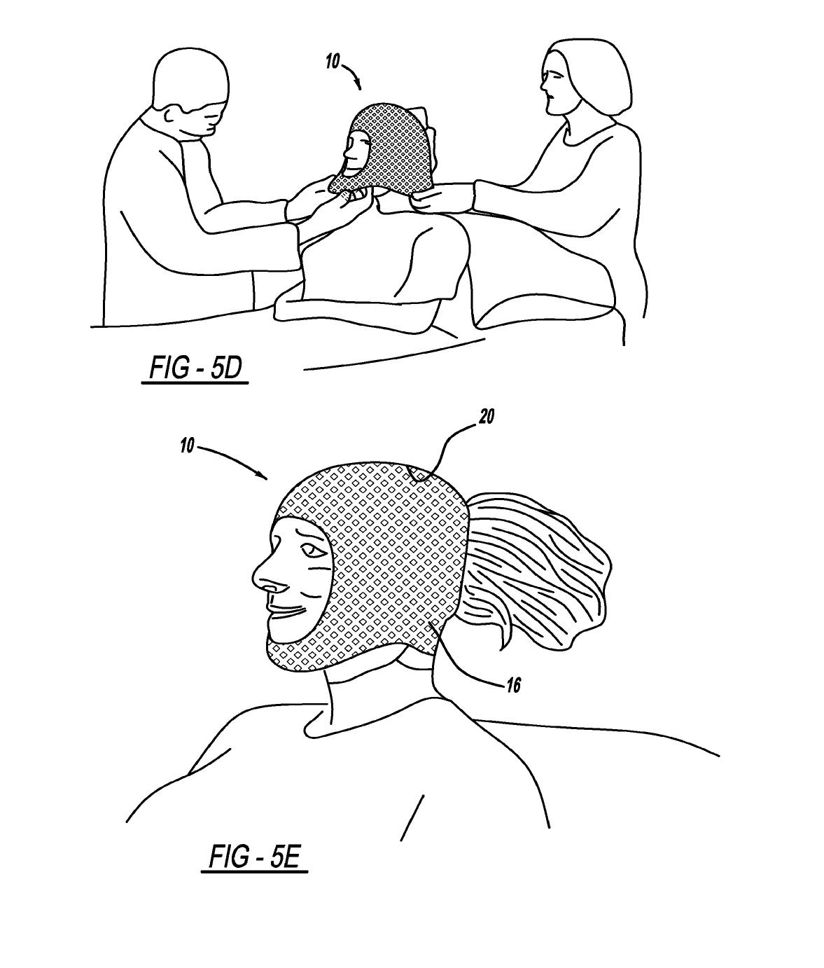

FIGS. 6A and 6B show the application of a cold compress to a patient's head (FIG. 6A), which is then held in place by the device of the present invention (FIG. 6B).

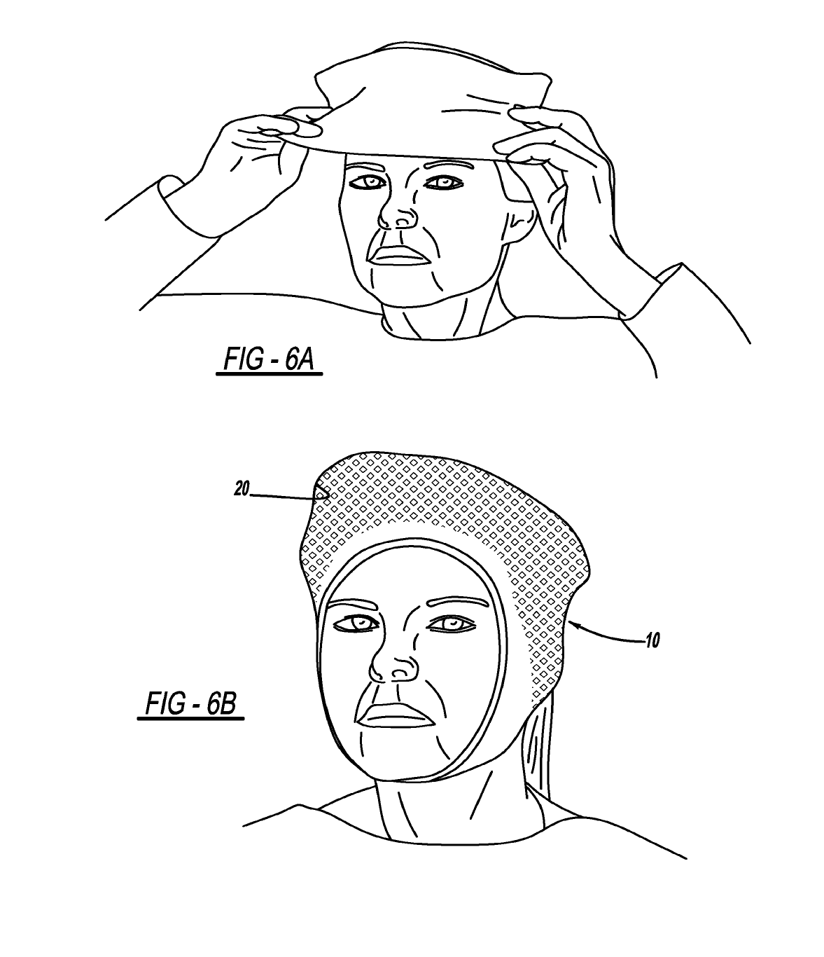

### DESCRIPTION OF THE CURRENT EMBODIMENT

Generally, the present invention relates to the use of a resilient, pliable, non-abrasive, non-absorbing material for forming a wearable article. The article is shown as 10 in the figures.

The article 10 can be an article of clothing, a wound care device, compression bandage, restraints, accessories, or any other device that would benefit from the qualities of the material disclosed herein. Examples of articles of clothing include, but are not limited to, socks, gloves, shirts, pants, undergarments, including both underwear and brassieres, to cover undergarments and/or prevent leakage of fluids, and dresses. The article 10 has an outer surface 20 and an inner surface 22 and can include a coating or other material on the inner surface 22 or impregnated in the materials itself, that can for example in the wound care use, aid or enhance the healing process. Such materials can include, but are not limited to, lotions, balms, medicine, ointments, compositions of matter (including herbs, honey, and other non-conventional medicines), or fabric/material. When additional materials are included in the article 10, the article 10 can be placed directly on the surface to be treated, the article can be applied either loosely or tightly depending on the intended use to create a barrier to limit access to the wound. The article 10 can also include padding or additional support features to further protect the wound or advance healing. Further, the article can include additional padding or cushioning at pressure points, which can also protect patients sensitive to pressure and touch. The article 10 can also include a bladder that can be filled with a fluid. For example, the bladder can be filled with either a hot or cold fluid that can retain the desired temperature.

The article of the present invention provides a greatly improved wound dressing wrap that 1) can be worn by patients throughout recovery, however lengthy; 2) can be manufactured in a range of sizes in order to provide a fully customizable fit for a wide range of patient body types; and 3) is constructed of materials that may be laundered and sanitized repeatedly without losing their original patient-friendly attributes.

The article 10 can also be used as a protective device. For example, the article 10 can be used as a pad on the arm of a wheelchair, or as an underarm pad for a crutch. Other similar uses can include rail pads for cribs, hospital beds, and other similar uses. Additionally, the article can be formed into a protective device for use in the home to protect against bumps and abrasions, such as for protecting toddlers and infants.

The article 10 can be also be used as a coverup to prevent fluids from either escaping or entering the protected area. For example, the article 10 can be used to protect a cast during showering.

Further, the article 10 can be used to maintain the positioning of medical devices. The article can be placed over the medical device to prevent the device from moving without causing additional abrasions on the wearer.

The article 10 can be in either tubular/sleeve, as shown, a tube or sleeve with a closed end (similar to a sock or glove), or sheet form (not shown). In sheet form, the ends of the sheet can be wrapped around the body part or item to be contained within the article and then either radio or sound waves can be used to meld or seal the ends of the sheet together (not shown). Thus, the radio/sound waves are used to alter the chemical structure of the sheet such that the ends of the sheet, when placed in contact with one another in the presence of radio/sound waves, adhere to one another and create a single piece of material.

The article 10 can also be fabricated into a tubular shape. The article 10 is hollow and has a first end 12 and a second end 14. The article 10 can be manufactured using methods known to those of skill in the art, which can include, but are not limited to, injection molding, extrusion, or dipping. The article 10 has substantial elasticity in the longitudinal and transverse directions, allowing the article 10 to return to its initial dimensions of length and width after any portion of the wrap is manually stretched in either direction. The medial portion of the wrap, as described below, can include holes 16 that permit substantial airflow through the article 10. The holes in the material can also function as finger/toe holes. The article 10 can also include a larger hole/opening 18 that exposes the dressing retained beneath the wrap. Alternatively, the article can also be made in a solid form. The holes can also be incorporated into the article post-manufacture.

The material used to form the article of the present invention is described in detail in U.S. Pat. No. 4,618,213, to Chen and incorporated herein by reference. The material is a gelatinous elastomer composition and can be prepared as disclosed in the '213 patent or the material can be modified to provide alternative characteristics, such as increased pliability or softness, wherein such modifications can be readily accomplished by one of skill in the art. The triblock copolymers employed in the present invention have the more general configuration A-B-A wherein each A is a crystalline polymer end block segment of polystyrene; and B is an elastomeric polymer center block segment of poly(ethylene-butylene). The poly(ethylene-butylene) and polystyrene portions are incompatible and form a two-phase system consisting of sub-micron domains of glassy polystyrene interconnected by flexible poly(ethylene-butylene) chains. These domains serve to crosslink and reinforce the structure. This physical elastomeric network structure is reversible, and heating the polymer above the softening point of polystyrene temporarily disrupts the structure, which can be restored by lowering the temperature.

Plasticizers particularly preferred for use in practicing the present invention are well known in the art, they include rubber processing oils such as paraffinic and naphthenic petroleum oils, highly refined aromatic-free paraffinic and naphthenic food and technical grade white petroleum mineral oils, and synthetic liquid oligomers of polybutene, polypropene, and polyterpene. The synthetic series process oils are high molecular weight oligomers, which are permanently fluid liquid monoolefins, isoparaffins or paraffins of moderate to high viscosity. Many such oils are known and commercially available.

The triblock copolymer component by itself lacks the desired contemplated properties; whereas, when the triblock copolymer (having styrene to ethylene and butylene ratio within the range contemplated in the instant invention) is combined with selected plasticizing oils with an average molecular weight of between about 200 to about 700, as determined by ebulliscopic methods, wherein, for most purposes, the oil constitutes about 300 to about 1,600 parts and more preferably about 350 to about 1,600 parts by weight of the triblock copolymer, that an extremely soft and highly elastic material is obtained. This transformation of the triblock copolymer structure in heated oil resulting in a composition having a gel rigidity of about 20 gram to about 700 gram Bloom and substantially without oil bleed out along with high tensile strength and elongation and other desirable combination of physical properties is unexpected. As used herein, the term “gel rigidity” in gram Bloom is determined by the gram weight required to depress a gel a distance of 4 mm with a piston having a cross-sectional area of 1 square centimeter at 23° C.

The composition of this invention can also contain small amounts of conventionally employed additives such as stabilizers, antioxidants, antiblocking agents, colorants, fragrances, flame retardants, and the like to an extend not affecting or decreasing the desired properties of the present invention. Additives useful in the compositions of the present invention include: Tetrakis [methylene 3,-(3′5′-di-tertbutyl-4″-hydroxyphenyl)propionate]methane, Octadecyl 3-(3″,5″-di-tert-butyl-4″-hydroxyphenyl)propionate, Distearyl-pentaerythritol-diphosphite, Distearyl-pentaerythritol-diphosphite, Tris(nonyl phenyl)phosphite, 2,6-di-tert-butyl-p-cresol, Dilaurylthiodiproprionate, Thiodiethylene bis-(3,5-tert-butyl-4-hydroxy) hydrocinnamate, (1,3,5-Trimethyl-2,4,6-tris [3,5-di-tert-butyl-4-hydroxybenzyl]benzene), 4,4″-Methylenebis(2,6-di-tert-butylphenol), Stearic Acid, Oleic Acid, Stearamide, Behenamide, Oleamide, Erucamide, N,N″-ethylenebisstearamide,N,N″-ethylenebisoleamide, Stearyl Erucamide, Erucyl Erucamide, Oleyl Palmitamide, Stearyl Stearamide, Erucyl Stearamide, metallic pigments (aluminum and brass flakes), TiO.sub.2, mica, fluorescent dyes and pigments, phosphorescent pigments, aluminatrihydrate, antimony oxide, molybdenum, silicone fluids, lake pigments, aluminates, ceramic pigments, ironblues, ultramarines, phthalocyanines, azo pigments, carbon blacks, silicon dioxide, silica, clay, feldspar, glass microspheres, barium ferrite, wollastonite and the like.

In accordance with the practice of the present invention, the aforementioned molecular weight range plasticizing oils can be used. Generally, plasticizing oils with average molecular weights less than about 200 and greater than about 700 may also be used.

The gelatinous elastomer compositions of the present invention are prepared by blending the components including other additives as desired at about 23° C. to about 100 degrees forming a paste like mixture and further heating said mixture uniformly to about 150° C. to 200° C. until a homogeneous molten blend is obtained. These components blend easily in the melt and a heated vessel equipped with a stirrer is all that is required.

The basis of this invention resides in the fact that a poly(styrene-ethylene-butylene-styrene) triblock copolymer having styrene end block to ethylene and butylene center block ratio within the contemplated range of from between 31:69 to 40:60 when blended in the melt with an appropriate amount of plasticizing oil makes possible the attainment of gelatinous elastomer compositions having a desirable combination of physical and mechanical properties, notably high elongation at break of at least 1,600%, ultimate tensile strength of about at least 8.times.10.sup.5 dyne/cm.sup.2, low elongation set at break of substantially not greater than about 2%, tear resistance of at least 5.times.10.sup.5 dyne/cm.sup.2, substantially about 100% snap back when extended to 1,200% elongation, and a gel rigidity of substantially not greater than about 700 gram Bloom. More specifically, the gelatinous compositions of the present invention exhibit one or more of the following properties. These are: (1) tensile strength of about 8.times.10.sup.5 dyne/cm.sup.2 to about 10.sup.7 dyne/cm.sup.2; (2) elongation of about 1,600% to about 3,000%; (3) elasticity modulus of about 10.sup.4 dyne/cm.sup.2 to about 10.sup.6 dyne/cm.sup.2; (4) shear modulus of about 10.sup.4 dyne/cm.sup.2 to about 10.sup.6 dyne/cm.sup.2 as measured with a 1, 2, and 3 kilogram load at 23° C.; (5) gel rigidity of about 20 gram Bloom to about 700 gram Bloom as measured by the gram weight required to depress a gel a distance of 4 mm with a piston having a cross-sectional area of 1 square cm at 23 ° C.; (6) tear propagation resistance of at least about 5.times.10.sup.5 dyne/cm.sup.2; (7) and substantially 100% snap back recovery when extended at a crosshead separation speed of 25 cm/minute to 1,200% at 23° C. Properties (1), (2), (3), and (6) above are measured at a crosshead separation speed of 25 cm/minute at 23 degrees C.

The gelatinous elastomer articles molded from the instant compositions have various additional important advantages in that they do not craze, creep, tear, crack, or rupture in flexural, extension, compression, or other deforming conditions of normal use; but rather the molded articles made from the instant composition possess the intrinsic properties of elastic memory enabling the articles to recover and retain its original molded shape after many extreme deformation cycles as compared to prior art triblock copolymer oil-extended compositions. In applications where low rigidity, high elongation, good compression set and excellent tensile strength are important, the instant compositions can be utilized.

The gelatinous elastomer compositions of the present invention are useful in low frequency vibration applications, such a viscoelastic layers in constrained-layer damping of mechanical structures and goods, as viscoelastic layers used in laminates for isolation of acoustical and mechanical noise, as viscoelastic layers used in wrappings, enclosures and linings to control sound, as compositions for use in shock and dielectric encapsulations of electrical and electronic components, as molded shape articles for use as therapeutic hand exercising grips, as articles for use as novel amusement toys, novel re-useable lint removers, optical lenses, light conductors such as pipes, tubes, cylinders, rods, prisms, cones, spheres and the like; other uses may include cladding for optical fibers.

In use, the article is molded or otherwise formed into the desired shape. The article can them be stretched as needed to be placed in position. For example, when the article is used for wound dressing care, the article is stretched and then positioned over the wound to maintain the dressings in proper position. By stretching the article there is less of a possibility of jarring or injuring the wound. Once in position, the article allows air to circulate and the article moves with, not against, the skin, thereby not further injuring or abrading the wound. Alternatively, the article can be rolled into a bagel/doughnut shape. The bagel/doughnut can then be stretched and placed about the wound area. Then, the bagel/doughnut can be unrolled to cover the wound area.

An alternative use of the article of the present invention is to retain an item in place. For example, the article can be used to retain skin/or and muscle in place for preoperative and postoperative surgery. In such use, the article is placed about the desired treatment location, then the skin is properly positioned underneath the article. The article can retain clotting materials and hold the skin and/or muscles in the proper position. Alternatively, the skin and/or muscle can be maneuvered into the proper position prior to positioning the article about the skin and/or muscle. In such use, the skin may have to be slightly re-adjusted after the article is positioned, in case any skin and/or muscle movement is detected.

The above description is that of the current embodiment of the invention. Various alterations and changes can be made without departing from the spirit and broader aspects of the invention. Any reference to elements in the singular, for example, using the articles “a,” “an,” “the” or “said,” is not to be construed as limiting the element to the singular.

1. A wound care device comprising:
a pliable tubular body, said tubular body being formed of a non-absorbing material, said tubular body having substantial elasticity in longitudinal and transverse directions of said tubular body.

2. The wound care device according to claim 1, wherein said tubular body has a plurality of holes formed therein.

3. The wound care device according to claim 1, wherein said non-absorbing material is impregnated with a second material for enhancing a healing process.

4. The wound care device according to claim 1, wherein said tubular body has one longitudinal end which is closed.

5. The wound care device according to claim 1, wherein said tubular body has material properties and is of a sufficient length to be rolled into and maintain a bagel/doughnut shape which can be unrolled after placement at a wound area.

6. The wound care device according to claim 1, wherein said tubular body has a tensile strength of 8×10⁵dyne/cm²to 10⁷dyne/cm² measured at a crosshead separation speed of 25 cm/minute at 23 degrees C.

7. The wound care device according to claim 1, wherein said tubular body has an elongation of 1,600% to 3,000% measured at a crosshead separation speed of 25 cm/minute at 23 degrees C.

8. The wound care device according to claim 1, wherein said tubular body has a modulus of elasticity of 10⁴dyne/cm²to 10⁶ dyne/cm² measured at a crosshead separation speed of 25 cm/minute at 23 degrees C.

9. The wound care device according to claim 1, wherein said tubular body has a shear modulus of 10⁴dyne/cm²to 10⁶ dyne/cm² as measured with a 1, 2, and 3 kilogram load at 23 degrees C.

10. The wound care device according to claim 1, wherein said tubular body has a gel rigidity of 20 gram Bloom to 700 gram Bloom as measured by the gram weight required to depress a gel a distance of 4 mm with a piston having a cross-sectional area of 1 square cm at 23 degrees C.

11. The wound care device according to claim 1, wherein said tubular body has a tear propagation resistance of at least 5×10⁵dyne/cm² measured at a crosshead separation speed of 25 cm/minute at 23 degrees C.

12. The wound care device according to claim 1, wherein said tubular body has a substantially 100% snap back recovery when extended at a crosshead separation speed of 25 cm/minute to 1,200% at 23 degrees.

13. The wound care device according to claim 1, wherein said tubular body has a tensile strength of 8×10⁶dyne/cm²to 10⁷dyne/cm² measured at a crosshead separation speed of 25 cm/minute at 23 degrees C., an elongation of 1,600% to 3,000% measured at a crosshead separation speed of 25 cm/minute at 23 degrees C., a modulus of elasticity of 10⁴dyne/cm²to 10⁶ dyne/cm² measured at a crosshead separation speed of 25 cm/minute at 23 degrees C., a shear modulus of 10⁴ dyne/cm² to 10⁶ dyne/cm² as measured with a 1, 2, and 3 kilogram load at 23 degrees C., a gel rigidity of 20 gram Bloom to 700 gram Bloom as measured by the gram weight required to depress a gel a distance of 4 mm with a piston having a cross-sectional area of 1 square cm at 23 degrees C., a tear propagation resistance of at least 5×10⁶dyne/cm² measured at a crosshead separation speed of 25 cm/minute at 23 degrees C., a substantially 100% snap back recovery when extended at a crosshead separation speed of 25 cm/minute to 1,200% at 23 degrees.

14. The wound care device according to claim 1, wherein said tubular body has a wall thickness, said wall thickness being thicker in areas which define padding for pressure points.
